(19) United States
(12) Patent Application Publication
(10) Pub. No.: US 2025/0204555 A1
(43) Pub. Date: Jun. 26, 2025

(54) FEED AND METHODS FOR CONTROLLING INTESTINAL TRACT PARASITIC WORM INFECTIONS IN MAMMALS

(71) Applicant: In the Bowl Animal Health, Nashville, TN (US)

(72) Inventors: Robin Readnour, Nashville, TN (US); Kevin E. Willard, Knightstown, IN (US); Joseph R. Winkle, Carmel, IN (US)

(21) Appl. No.: 19/077,550

(22) Filed: Mar. 12, 2025

Related U.S. Application Data
(63) Continuation of application No. PCT/US2023/074321, filed on Sep. 15, 2023.
(60) Provisional application No. 63/407,146, filed on Sep. 15, 2022.

(51) Int. Cl. A23K 50/40; A23K 20/111; A23K 20/137

(52) U.S. Cl. CPC A23K 50/40; A23K 20/111; A23K 20/137

(57) ABSTRACT

An oral animal feed and a method of preventing and/or controlling parasitic worms in an mammal in need thereof by orally administering to the mammal, for example a canine, a feed or a composition comprising an effective amount of an anthelmintic that is delivered on a daily or a substantially daily basis for an effective time to thereby cause the amount of the anthelmintic in the mammal's digestive tract to rise to and be maintained at a therapeutically effective level for controlling parasitic worms.

### RELATED APPLICATIONS

This application claims priority to U.S. Provisional Patent Application No. 63/407,146, filed Sep. 15, 2022, and PCT Application No. PCT/US2023/74321, filed on Sep. 15, 2023, the entire content of which are hereby incorporated herein by reference.

### BACKGROUND AND SUMMARY

The teachings of this disclosure generally relate to a method of administering an active material to control parasitic worms in mammals.

There are five main types of parasitic worms, or helminths, that commonly infect domestic dogs and other mammals: ascarid worms, tapeworms, hookworms, whipworms and heartworms. Helminths can cause serious health problems, including gastro-intestinal issues, abdominal pain, weight loss, dehydration, anemia, pneumonia and death in severe cases. Some of these intestinal helminth species are also of zoonotic importance by causing disease in humans. Thus, it is essential that dogs and cats kept as pets receive appropriate veterinary care, including treatment to prevent or treat infections with these various helminth parasites.

Treatments currently available for controlling helminth infections in animals are known to achieve varying degrees of success. Issues that contribute to ineffective control of parasitic worms in, for example, dogs and cats include improper oral dosing that may lead to further anthelmintic resistance and vomiting caused by the high doses required for some anthelmintics to achieve effectiveness.

Embodiment 1: A medicated animal feed for controlling intestinal helminths in a mammal, comprising:
a feedstuff; and
a dose of at least one anthelmintic that:
   (i) disrupts cellular integrity in intestinal helminths and is selected from the group consisting of benzimidazoles, salicylanilides, and substituted phenyls, and/or
   (ii) causes paralysis in intestinal helminths and is selected from the group consisting of tetrahydropyrimidines, emodepside, and monepantel;
wherein the at least one anthelmintic is an integral part of the medicated animal feed and the effective time to achieve a therapeutically effective level of the anthelmintic in the mammal's digestive tract is selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

Embodiment 2: The medicated animal feed according to embodiment 1, wherein the feedstuff is selected from the group consisting of: a kibble, a wet preparation, a treat, and a snack.

Embodiment 3: The medicated animal feed according to embodiments 1-2, wherein the medicated feed is suitable to feed to the mammal on a daily or substantially daily basis over the period of time.

Embodiment 4: The medicated animal feed according to embodiments 1-3, wherein the mammal is selected from a group consisting of: canines and felines.

Embodiment 5: The medicated animal feed according to embodiments 1-4, wherein the mammal is a dog.

Embodiment 6: The medicated animal feed according to embodiments 1-4, wherein the mammal is a cat.

Embodiment 7: The medicated animal feed according to embodiments 1-6, wherein the intestinal helminth is at least one intestinal helminth selected from the group consisting of: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

Embodiment 8: The medicated animal feed according to embodiments 1-7, wherein the medicated feed reduces the intestinal helminth count in a mammal by at least 50%, by at least 75%, by at least 80%, by at least 88%, by at least 95%, or by 100% as compared to a mammal not receiving the medicated feed.

Embodiment 9: The medicated animal feed according to embodiments 1-8 wherein the medicated feed protects the mammal from intestinal helminths for a period of at least 7 days after the last administration of medicated feed.

Embodiment 10: The medicated animal feed according to embodiments 1-9, wherein the anthelmintic is pyrantel or a therapeutically acceptable derivative, salt or metabolite thereof, and wherein the amount of pyrantel in the medicated feed is such that the total therapeutic dose of pyrantel does not exceed about 50.0 mg of pyrantel per kg of the mammal's body mass (mg/kg) when administered to the mammal in a plurality of doses within a period of time of about 30 days.

Embodiment 11: The medicated animal feed according to embodiment 10, wherein the anthelmintic is pyrantel pamoate.

Embodiment 12: The medicated animal feed according to embodiments 10-11, wherein the daily dose of pyrantel in the medicated feed is selected from the group consisting of: about 0.1 mg/kg, about 0.25 mg/kg, about 0.5 mg/kg, and about 1.0 mg/kg.

Embodiment 13: The medicated animal feed according to embodiments 10-12, wherein the cumulative dose of pyrantel administered to the mammal over 30 days is selected from the group consisting of: about 3 mg/kg, about 7.5 mg/kg, about 15 mg/kg and about 30 mg/kg.

Embodiment 14: The medicated animal feed according to embodiments 1-9 wherein the anthelmintic is monepantel or a therapeutically acceptable derivative, salt or metabolite thereof, and wherein the amount of monepantel in the medicated feed is such that the total therapeutic dose of monepantel does not exceed about 25.0 mg of monepantel per kg of the mammal's body mass (mg/kg) when administered to the mammal in a plurality of doses within a period of time of about 30 days.

Embodiment 15: The medicated animal feed according to embodiment 14, wherein the daily dose of monepantel in the medicated feed is selected from the group consisting of: about 0.05 mg/kg, about 0.125 mg/kg, about 0.25 mg/kg, about 0.5 mg/kg, about 0.83 mg/kg.

Embodiment 16: The medicated animal feed according to embodiments 14-15, wherein the cumulative dose of monepantel administered to the mammal over 30 days is selected from the group consisting of: about 1.5 mg/kg, about 3.75 mg/kg, about 7.5 mg/kg and about 25.0 mg/kg.

Embodiment 17: The medicated animal feed according to embodiments 1-8, wherein the anthelmintic in the medicated feed that disrupts cellular integrity in the intestinal helminths is a benzimidazole.

Embodiment 18: The medicated animal feed according to embodiment 17, wherein the anthelmintic is fenbendazole or a therapeutically acceptable derivative, salt or metabolite thereof, and wherein the amount of fenbendazole in the medicated feed is such that the total therapeutic dose of fenbendazole does not exceed about 300 mg of fenbendazole per kg of the mammal's body mass (mg/kg) when administered to the mammal in a plurality of doses within a period of time of about 30 days.

Embodiment 19: The medicated animal feed according to embodiment 18, wherein the daily dose of fenbendazole in the medicated feed is selected from the group consisting of: about 0.1 mg/kg, about 0.25 mg/kg, about 0.5 mg/kg, about 1.0 mg/kg, about 2.5 mg/kg, about 5.0 mg/kg, and about 10 mg/kg.

Embodiment 20: The medicated animal feed according to embodiment 19, wherein the cumulative dose of fenbendazole administered to the mammal over 30 days is selected from the group consisting of: about 3.0 mg/kg, about 7.5 mg/kg, about 15.0 mg/kg, about 30.0 mg/kg, about 75.0 mg/kg, about 150.0 mg/kg, and about 300.0 mg/kg.

Embodiment 21: The medicated animal feed according to embodiments 1-20, wherein the medicated animal feed further comprises a therapeutically effective amount of praziquantel or a therapeutically acceptable derivative, salt or metabolite thereof.

Embodiment 22: A method for controlling intestinal helminths in a mammal, comprising the steps of:
providing the medicated feed according to embodiments 1-21; and
administering said feed to a mammal on a daily or a substantially daily basis.

Embodiment 23: The method according to embodiment 22, wherein the mammal is selected from the group consisting of: canines and felines.

Embodiment 24: The method according to embodiments 22-23, wherein the mammal is a dog.

Embodiment 25: The method according to embodiments 22-24, wherein the mammal is a cat.

Embodiment 26: The method according to embodiments 22-25, wherein the intestinal helminth is at least one intestinal helminth selected from the group consisting of: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

Embodiment 27: The method according to embodiments 22-26, wherein the medicated feed is administered over a period selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

Embodiment 28: A composition for controlling intestinal helminths in a mammal, comprising:
at least one anthelmintic that:
   (i) disrupts cellular integrity in intestinal helminths and is selected from the group consisting of benzimidazoles, salicylanilides, and substituted phenyls, and/or
(ii) causes paralysis in intestinal helminths and is selected from the group consisting of tetrahydropyrimidines, emodepside, and monepantel;
wherein the effective time to achieve a therapeutically effective level of the anthelmintic in the mammal's digestive tract is selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

Embodiment 29: The composition according to embodiment 28, wherein the composition is selected from the group consisting of: a kibble, a wet feed, a tablet, a capsule, a suspension, a solution, a salve, a paste, a treat, and a snack.

Embodiment 30: The composition according to embodiments 28-29, wherein the composition is suitable to administer to the mammal on a daily or substantially daily basis over the period of time.

Embodiment 31: The composition according to embodiments 28-30, wherein the mammal is selected from a group consisting of: canines and felines.

Embodiment 32: The composition according to embodiments 28-31, wherein the mammal is a dog.

Embodiment 33: The composition according to embodiments 28-31, wherein the mammal is a cat.

Embodiment 34: The composition according to embodiments 28-33, wherein the intestinal helminth is at least one intestinal helminth selected from the group consisting of: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

Embodiment 35: The composition according to embodiments 28-34, wherein the composition reduces the intestinal helminth count in the mammal by at least 50%, by at least 75%, by at least 80%, by at least 88%, by at least 95%, or by 100% as compared to a mammal not receiving the composition.

Embodiment 36: The composition according to embodiments 28-35 wherein the composition protects the mammal from intestinal helminths for a period of at least 7 days after the last administration.

Embodiment 37: The composition according to embodiments 28-36, wherein the anthelmintic is pyrantel or a therapeutically acceptable derivative, salt or metabolite thereof, and wherein the amount of pyrantel in the composition is such that the total therapeutic dose of pyrantel does not exceed about 50.0 mg of pyrantel per kg of the mammal's body mass (mg/kg) when administered to the mammal in a plurality of doses within a period of time of about 30 days.

Embodiment 38: The composition according to embodiment 37, wherein the anthelmintic is pyrantel pamoate.

Embodiment 39: The composition according to embodiments 37-38, wherein the daily dose of pyrantel in the medicated feed is selected from the group consisting of: about 0.1 mg/kg, about 0.25 mg/kg, about 0.5 mg/kg, and about 1.0 mg/kg.

Embodiment 40: The composition according to embodiments 37-39, wherein the cumulative dose of pyrantel administered to the mammal over 30 days is selected from the group consisting of: about 3.0 mg/kg, about 7.5 mg/kg, about 15 mg/kg and about 30 mg/kg.

Embodiment 41: The composition according to embodiments 28-36 wherein the anthelmintic is monepantel or a therapeutically acceptable derivative, salt or metabolite thereof, and wherein the amount of monepantel in the composition is such that the total therapeutic dose of monepantel does not exceed about 25.0 mg of monepantel per kg of the mammal's body mass (mg/kg) when administered to the mammal in a plurality of doses within a period of time of about 30 days.

Embodiment 42: The composition according to embodiment 41, wherein the daily dose of monepantel in the composition is selected from the group consisting of: about 0.05 mg/kg, about 0.125 mg/kg, about 0.25 mg/kg, about 0.5 mg/kg and about 0.83 mg/kg.

Embodiment 43: The composition according to embodiments 41-42, wherein the cumulative dose of monepantel administered to the mammal over 30 days is selected from the group consisting of: about 1.5 mg/kg, about 3.75 mg/kg, about 7.5 mg/kg, about 15 mg/kg and about 25.0 mg/kg.

Embodiment 44: The composition according to embodiments 28-36, wherein the anthelmintic in the composition that disrupts cellular integrity in the intestinal helminths is a benzimidazole.

Embodiment 45: The composition according to embodiment 44, wherein the anthelmintic is fenbendazole or a therapeutically acceptable derivative, salt or metabolite thereof, and wherein the amount of fenbendazole in the composition is such that the total therapeutic dose of fenbendazole does not exceed about 300 mg of fenbendazole per kg of the mammal's body mass (mg/kg) when administered to the mammal in a plurality of doses within a period of time of about 30 days.

Embodiment 46: The composition according to embodiment 45, wherein the daily dose of fenbendazole in the composition is selected from the group consisting of: about 0.1 mg/kg, about 0.25 mg/kg, about 0.5 mg/kg, about 1.0 mg/kg, about 2.5 mg/kg, about 5.0 mg/kg, and about 10 mg/kg.

Embodiment 47: The composition according to embodiment 45-46, wherein the cumulative dose of fenbendazole administered to the mammal over 30 days is selected from the group consisting of: about 3.0 mg/kg, about 7.5 mg/kg, about 15.0 mg/kg, about 30.0 mg/kg, about 75.0 mg/kg, about 150.0 mg/kg, and about 300.0 mg/kg.

Embodiment 48: The composition according to embodiments 1-20, wherein the composition further comprises a therapeutically effective amount of praziquantel or a therapeutically acceptable derivative, salt or metabolite thereof.

Embodiment 49: A method for controlling intestinal helminths in a mammal, comprising the steps of:
providing the composition according to embodiments 28-48; and
administering said composition to a mammal on a daily or a substantially daily basis.

Embodiment 50: The method according to embodiment 49, wherein the mammal is selected from the group consisting of: canines and felines.

Embodiment 51: The method according to embodiments 49-50, wherein the mammal is a dog.

Embodiment 52: The method according to embodiments 49-51, wherein the mammal is a cat.

Embodiment 53: The method according to embodiments 49-52, wherein the intestinal helminth is at least one intestinal helminth selected from the group consisting of: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

Embodiment 54: The method according to embodiments 49-53, wherein the composition is administered over a period selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

Embodiment 55: A medicated feed according to embodiments 1-21 for use in a method for controlling intestinal helminths in a mammal, the method comprising the steps of:
providing the medicated feed; and
administering said feed to a mammal on a daily or a substantially daily basis.

Embodiment 56: The medicated feed for use in the method according to embodiment 55, wherein the mammal is selected from the group consisting of: canines and felines.

Embodiment 57: The medicated feed for use in the method according to embodiments 55-56, wherein the mammal is a dog.

Embodiment 58: The medicated feed for use in the method according to embodiments 55-56, wherein the mammal is a cat.

Embodiment 59: The medicated feed for use in the method according to embodiments 55-58, wherein the intestinal helminth is at least one intestinal helminth selected from the group consisting of: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

Embodiment 60: The medicated feed for use in the method according to embodiments 55-59, wherein the medicated feed is administered over a period selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

Embodiment 61: A composition according to embodiments 28-48 for use in a method for controlling intestinal helminths in a mammal, the method comprising the steps of: providing the composition; and administering said composition to a mammal on a daily or a substantially daily basis.

Embodiment 62: The composition for use in the method according to embodiment 61, wherein the mammal is selected from the group consisting of: canines and felines.

Embodiment 63: The composition for use in the method according to embodiments 61-62, wherein the mammal is a dog.

Embodiment 64: The composition for use in the method according to embodiments 61-62, wherein the mammal is a cat.

Embodiment 65: The composition for use in the method according to embodiments 61-64, wherein the intestinal helminth is at least one intestinal helminth selected from the group consisting of: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

Embodiment 66: The composition for use in the method according to embodiments 61-65, wherein the composition is administered over a period selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

### BRIEF DESCRIPTION OF THE FIGURES

FIG. 1. Histogram showing the percent efficacy calculated as: [AM fecal eggs CONTROL-AM fecal eggs TREATED]/AM fecal eggs CONTROL at 16, 19, 22, 25 28 or 30 days after initial treatment with one of the following: 0.5 mg/kg Pyrantel pamoate (horizontal hash), 1.0 mg/kg Pyrantel pamoate (right hash), 5 mg/kg Fenbendazole (stippled) and 10 mg/kg Fenbendazole (vertical hash).

FIG. 2. Histogram showing the percent efficacy calculated as: [AM fecal eggs CONTROL-AM fecal eggs TREATED]/AM fecal eggs CONTROL at 40, 45, 50, 52, or 60 days after initial treatment with one of the following; 0.25 mg/kg Pyrantel pamoate (horizontal hash), 0.5 mg/kg Pyrantel pamoate (right hash), 1.0 mg/kg Pyrantel pamoate (stippled), 2.5 mg/kg Fenbendazole (vertical hash), 5 mg/kg Fenbendazole (solid), or 10 mg/kg Fenbendazole (left hash).

### DESCRIPTION

Surprisingly, this disclosure teaches that in some instances oral treatments with some anthelmintics can provide improved control over parasitic worm infections in mammals when the anthelmintics are orally administered to a mammal in smaller, more frequent/chronic doses than the doses currently recorded for administration on a monthly or quarterly basis. In some embodiments, the administration is discussed below as being integrally combined with a feed. However, it is also contemplated that an anthelmintic may be administered by itself or in a dosage form other than as an integral part of a feed, such as a chew, tablet, liquid, gel or other suitable form for oral administration. Advantageously, in some instances, by using smaller, more frequent doses, less total anthelmintic is required over the same time period to control parasitic worm infections.

Advantageously, the total amount of anthelmintic required for a therapeutically effective once-monthly dose can be reduced by as much as 80 percent for some anthelmintics by converting to daily administration. From a practical perspective, formulating a medicinal feed produces at least two problems: (1) creating a homogenous feed; and (2) analytical control as it requires testing for a very small dose of a more potent anthelmintic, which may be difficult to accomplish. The analytical matrix from feeds can be quite complex and difficult to assay. Assays may be in the parts per million to billion range for some needed doses and feed concentrations. Thus, it is possible that one of skill in the art may opt to increase the daily dose of a more potent anthelmintic such that the total of the daily doses over the course of one month equals the prior art once-monthly dose or is even higher, for example, 200% of the prior art once-monthly dose. This may be done to help ensure homogeneity as well as increase assay accuracy and decrease analytical variability when administering the dose as part of a feed while still producing a net therapeutic dose that is some multiple less than the currently recommended monthly therapeutic dose.

The methods and compositions taught herein have the further advantage of encouraging compliance because the smaller doses of an anthelmintic can be incorporated into a feed or formulations administered as a chew, tablet, liquid, gel or other suitable form for oral administration. In some embodiments, a daily dose of anthelmintic can be added or combined with the feed. Since owners naturally follow a daily feeding regimen in any event, this makes it less likely that owners will forget or neglect to administer the anthelmintic. Thus, this disclosure provides methods for prolonged control of parasitic worm in a safer and more effective manner than that achieved with previously known treatment methodologies. All the owner need remember is to feed their pet daily as they normally would and as described in this embodiment and concomitantly adding a daily dose of anthelmintic.

Several classes of anthelmintically active compounds are known, including, for example: the macrocyclic lactones, such as abamectin, doramectin, emamectin, eprinomectin, ivermectin, milbemycin, moxidectin, nemadectin, selamectin; the benzimidazoles and probenzimidazoles, such as albendazole, albendazole-sulphoxide, cambendazole, cyclobendazole, febantel, fenbendazole, flubendazole, mebendazole, netobimin, oxfendazole, oxibendazole, parbendazole, thiabendazole, thiophanate, triclabendazole; the cyclooctadepsipeptides, such as emodepside, PF1022; the aminoacetonitrile derivatives, such as monepantel; the tetrahydropyrimidines, such as morantel, pyrantel, oxantel; the imidazothiazoles, such as butamisole, levamisole, tetramisole; the salicylanilides, such as bromoxanide, brotianide, clioxanide, closantel, niclosamide, oxyclozanide, rafoxanide, tribromsalan; the paraherquamides, such as derquantel, paraherquamide; the aminophenylamidines, such as amidantel, deacylated amidantel (dAMD), tribendimidine; the organophosphates, such as coumaphos, crufomate, dichlorvos, haloxone, naphthalofos, trichlorfon; the substituted phenyls, such as bithionol, disophenol, hexachlorophene, niclofolan, meniclopholan, nitroxynil; the piperazinones, such as praziquantel, epsiprantel; and other classes, which include such examples as amoscanate, bephenium, bunamidine, clonazepam, clorsulon, diamfenetid, dichlorophen, diethylcarbamazine, emetine, hetolin, hycanthone, lucanthone, Miracil, mirasan, niclosamide, niridazole, nitroxynil, nitroscanate, oltipraz, omphalotin, oxamniquin, paromomycin, piperazine and resorantel.

Anthelmintics of particular interest for controlling parasitic worm infections as discussed in some embodiments in mammals are benzimidazoles and aminoacetonitrile derivatives.

Some benzimidazoles are anthelmintics that disrupt cellular integrity in helminths. They are bicyclic compounds having fused rings of the aromatic compounds benzene and imidazole. One example of a benzimidazole is fenbendazole (methyl N-(6-phenylsulfanyl-1H-benzimidazol-2-yl) carbamate), shown below:

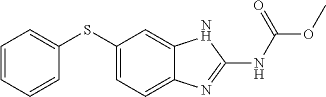 [SMILES: COC(=O)Nc1nc2ccc(Sc3ccccc3)cc2[nH]1]

Pyrantel and its various salt forms, members of the tetrahydropyrimidine family of compounds, are known veterinary anthelmintic drugs that are also used in human medicine. Pyrantal pamoate, one example of a tetrahydropyrimidine, is reported to have a mode of action that involves the nicotinic acetylcholine receptor that is the putative target in some helminths, is shown below:

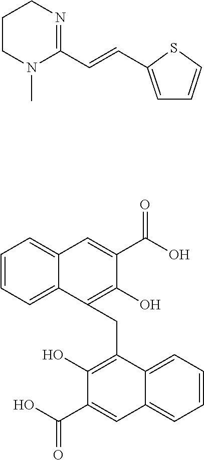 [SMILES: CN1CCCN=C1/C=C/c1cccs1 ; O=C(O)c1cc2ccccc2c(Cc2c(O)c(C(=O)O)cc3ccccc23)c1O]

The aminoacetonitrile derivatives, such as monepantel (N-[(2S)-2-cyano-1-[5-cyano-2-(trifluoromethyl) phenoxy]propan-2-yl]-4-(trifluoromethylsulfanyl)benzamide), are thought to act by interfering with parasite muscle function. Monepantel, its salts, derivatives, metabolites and any biologically active enantiomers thereof are aminoacetonitriles that may be suitable for use with this disclosure.

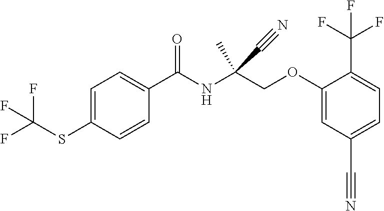 [SMILES: C[C@](C#N)(COc1cc(C#N)ccc1C(F)(F)F)NC(=O)c1ccc(SC(F)(F)F)cc1]

More particularly, anthelmintics with a half-life of about at least 1 day may be especially well-suited for the methods and formulations of this disclosure.

The formulations, or feeds, and methods of this disclosure may further include, in combination with the anthelmintic, one or more other active drug substances having therapeutic efficacy. Such active substances include agents efficacious against parasitic worms and/or associated carriers of parasitic worms. Active substances may include, for example, praziquantel, isoxazolines, foramidines, spinosyns, macrocyclic lactones, and insect growth regulators (including chitin synthesis inhibitors, juvenile hormone analogs, and juvenile hormones).

For example, praziquantel (2-(cyclohexanecarbonyl)-3,6,7,11b-tetrahydro-1H-pyrazino[2,1-a]isoquinolin-4-one), shown below, or a therapeutically acceptable salt, metabolite or derivative thereof, may be included in the inventive formulations and compositions.

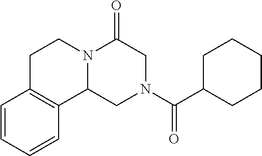 [SMILES: O=C(C1CCCCC1)N1CC(=O)N2CCc3ccccc3C2C1]

The methods of this disclosure are carried out by administering the anthelmintic to the mammal in small, frequent doses. To facilitate routine dosing, the anthelmintic administration may be carried out using a feed or chew. A number of different feeds are envisioned, provided the manufacturing process(es) and feed compositions do not have deleterious effects related to chemical stability, efficacy and safety on the anthelmintic and, if applicable, other active substances. For example, feeds and snacks, chews, treats or supplemental feeds in the broad categories of dry, semi-moist, canned-retorted feeds or fresh refrigerated feeds may be adapted for use with this disclosure. The mammal receives a maintenance quantity of anthelmintic by consuming the feed product on a weekly, semi-weekly or daily basis.

By incorporating smaller doses of an anthelmintic into an animal feed composition and administering it at an effective rate (most preferably daily), the levels of the anthelmintic in the mammal's digestive tract can be maintained at a level effective to treat and prevent further worm infections. By contrast, when an anthelmintic is orally administered in larger doses at lower frequency, e.g., a single treatment of a large dose that is administered just once as a single therapeutic treatment, the level of the anthelmintic in the digestive tract may be sufficient to treat an active infection, but may not be effective to prevent a subsequent infection. As disclosed herein, a plurality of small doses administered over a period of time are at least as effective as a large dose administered in a single treatment, as constitutes the current standard of care for treating worm infections in the gastrointestinal tract. Surprisingly, the cumulative amount of the anthelmintic administered in a plurality of small doses may be substantially lower, but still as effective as a single large dose given less frequently, e.g., monthly.

All ratios, percentages, and parts discussed herein are “by weight” unless otherwise specified.

Doses formulated for daily administrations may include tablets, capsules, salves, liquids, and pastes.

The terms “controlling a parasitic worm infection” and “controlling a helminth infection” refer to preventing, treating, minimizing or eliminating an infection by parasitic worms or helminths in a mammal.

The term “controlling a parasitic worm infection” refers to preventing, treating, minimizing or eliminating an infection by parasitic worms in a mammal.

The terms “parasitic worms” and “helminths” are used interchangeably to refer to members of the phyla Annelida, Platyhelminthes, Nematoda and Acanthocephala. The terms “parasitic worms” and “helminths” include the egg, larval and adult stages of development. For example, “parasitic worms” or “helminths” may include but are not limited to the following: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

The term “canine” refers to any member of the genus Canis, which includes such species as wolves, dogs, coyotes and jackals.

The term “feline” refers to any member of the subfamily Felidae, which includes such species as the domestic cat, bobcats, wildcats, ocelots, members of the genus lynx, Pallas's cat and cougars.

In carrying out the methods of this disclosure, a “feed” is an animal feed, snack, treat or other supplemental feed that may be administered daily or substantially daily. By using different forms of feed, e.g., kibble and treats, a pet owner may vary the mammal's meals and snacks from time to time while still conveniently administering a daily dose of anthelmintic.

The term “chew” refers to a treat that usually has flavor and aromatic properties that are appealing to a mammal, but typically has no nutritional value. In carrying out the methods of this disclosure, a “feed” and/or a “chew” may be used interchangeably.

The term “effective time,” also referred to herein as “effective duration,” for the purposes of this disclosure includes at least the duration of feed administration needed to bring the level of anthelmintic in the mammal's digestive tract to a sufficiently high level for controlling parasitic worms, i.e., a “therapeutically effective” level. In some embodiments, the effective time may be as little as three days. In other instances, the effective time may be seven days or fifteen days or longer. As discussed below, the effective time will vary based on how frequently the feed or anthelmintic is administered.

As just alluded, the “effective time” will vary as a function of the frequency at which the feed is administered. The term “effective frequency” as used herein means the number of feedings over a given time that produce a therapeutically effective concentration of anthelmintic in the mammal's gastrointestinal tract. In all events, the term “effective frequency” as used herein contemplates multiple feedings including the anthelmintic per month. One of skill in the art will appreciate that the anthelmintic may be administered in a range of frequencies. For example, the anthelmintic may be administered at a frequency of daily, every other day, every third day, once per week or even at inconsistent time intervals.

Further, as discussed above, the effective frequency may affect the duration required to obtain a therapeutically effective level of anthelmintic in the mammal's digestive tract. By way of example, if the mammal were being fed an anthelmintic composition daily, the duration of feed administration required to achieve a therapeutically effective level of anthelmintic in the mammal's gastrointestinal tract, and thus the “effective time,” would be comparatively less than if the mammal were being fed the anthelmintic composition only once or twice per week.

Further, the effective frequency is influenced by the amount of the daily dose in mg/kg of body weight of the mammal. Particularly, at slightly higher daily doses, missed doses have less of an impact on efficacy.

Further, the effective frequency is influenced by the duration of treatment. In the initial stages, e.g., before the amount of anthelmintic in the mammal's digestive tract has reached a therapeutically effective level, the animal feed may need to be administered more often than would be necessary after a longer period of use, i.e., once a therapeutically effective level is obtained.

For purposes of this disclosure, “substantially daily” means a sufficiently regular basis such that the anthelmintic concentration in the mammal's digestive tract rises to and remains at a therapeutically effective level. For example, the disclosed daily feed composition can preferably be fed to a mammal every day indefinitely. However, as a practical matter, there are many reasons why days may be missed or skipped periodically. For example, the mammal may be ill or the owner may run out of the daily medicated feed composition. The disclosed method is robust enough that the mammal will still be protected from parasitic worms to some extent even with occasional interruptions in daily feeding of the medicated animal feed composition. In carrying out the method of this disclosure, the term “substantially daily” includes at least 10 days per month, more preferably at least 15 days per month, still more preferably at least 20 days per month. All of these feeding frequencies, whether they be, e.g., three times per week, every other day or daily, fit under the umbrella of substantially daily provided that they promote the anthelmintic reaching and maintaining a therapeutically effective level of the anthelmintic in the mammal's gastrointestinal tract.

The term “therapeutically effective” means that the dose of an anthelmintic or a physiologically acceptable derivative thereof, or a metabolite thereof, is sufficient to control the helminth infection better than if no drug were present. The anthelmintic or a physiologically acceptable derivative thereof, or a metabolite thereof, may be present on its own or with one or more additional active substances. Preferably it controls the helminth infection at around at least 50% better than if no drug were present, and more preferably it controls the helminth infection at about at least 90% better than if no drug were present.

In carrying out the methods of this disclosure, an effective or therapeutically effective amount of an anthelmintic is administered orally to the mammal. The term “effective amount” or “therapeutically effective amount” refers to the amount needed to control the helminth infection. As those skilled in the art will understand, this amount will vary depending upon a number of factors. These factors include, for example, the type of mammal being treated and its weight and general physical condition.

Anthelmintics vary in potency. Thus, the effective amount of anthelmintic may be calculated for each particular anthelmintic used in some of the methods according to this disclosure. In general, the effective amount for a daily dose of an anthelmintic may be in the range of about 12.5%-90% of the approved label dose for said anthelmintic divided by length of the dosing/retreatment interval (e.g., the dosage divided by 30 for a product administered once per month). One of skill in the art will recognize that a higher dose of, e.g., 90%-200% of the approved label dose for said anthelmintic may be selected for reasons such as, but not limited to, manufacturability, ease of testing and analysis, etc. In some instances the particular dose selected may be sufficient to raise the mammal's concentration of said anthelmintic in the digestive tract to a therapeutically effective level within about 7 days of substantially daily administrations, more preferably within about 5 days of substantially daily administrations, most preferably within about 3 days of substantially daily administrations.

While this disclosure describes concentrations of anthelmintic in terms of feeds such as kibble, it also contemplates administration using other dosage forms, such as treats or chews. It is also contemplated that the anthelmintic may be administered by itself or in a tablet, liquid, gel or other suitable form for oral administration. One of skill in the art will appreciate that the concentration of anthelmintic will vary according to the particular dosage form. For example, where the animal feed is a treat, the concentration of anthelmintic in the treat will be greater than, e.g., the concentration of anthelmintic in a kibble. For example, if the daily dose of anthelmintic based on the weight of the mammal is 10 mg, then a typical 5 g treat may contain about 0.002 percent anthelmintic (by weight). Since the amount of kibble consumed in a day is more than 5 g, the percent anthelmintic in kibble will be smaller.

For example, an effective amount of pyrantel pamoate may be a daily or a substantially daily dose of from about 0.1 to about 1.0 mg of pyrantel pamoate/kg of body weight of the mammal. Alternatively, an effective amount of pyrantel pamoate may be a daily or a substantially daily dose of from about 0.1 to about 0.5 mg of pyrantel pamoate/kg of body weight of the mammal. Alternatively, the effective amount of pyrantel pamoate may be a daily or a substantially daily dose of from about 0.25 to about 0.5 mg/kg of body weight of the mammal. Alternatively, the effective amount of pyrantel pamoate may be a daily or a substantially daily dose of from about 0.1 to about 0.25 mg of pyrantel pamoate/kg of body weight of the mammal.

Animal feeds will typically contain from about 0.0001 to about 0.09 percent of pyrantel pamoate (by weight) in the feed. Preferably between about 0.0005 to about 0.06 percent of pyrantel pamoate (by weight) in the feed. Most preferably between about 0.0009 to about 0.03 percent of pyrantel pamoate component or components (by weight) in the feed.

In another example, an effective amount of fenbendazole may be a daily or a substantially daily dose of from about 0.5 to about 10.0 mg of fenbendazole/kg of body weight of the mammal. Alternatively, an effective amount of fenbendazole may be a daily or a substantially daily dose of from about 1.0 to about 5.0 mg of fenbendazole/kg of body weight of the mammal. Alternatively, the effective amount of fenbendazole may be a daily or a substantially daily dose of from about 2.5 to about 5.0 mg of fenbendazole/kg of body weight of the mammal. Alternatively, the effective amount of fenbendazole may be a daily or a substantially daily dose of from about 0.5 to about 2.5 mg of fenbendazole/kg of body weight of the mammal.

Animal feeds will typically contain from about 0.001 to about 0.12 percent of fenbendazole (by weight) in the feed. Preferably between about 0.003 to about 0.09 percent of fenbendazole (by weight) in the feed. Most preferably between about 0.008 to about 0.05 percent of fenbendazole component or components (by weight) in the feed.

In another example, an effective amount of monepantel may be a daily or a substantially daily dose of from about 0.05 to about 0.83 mg of monepantel/kg of body weight of the mammal. Alternatively, an effective amount of monepantel may be a daily or a substantially daily dose of from about 0.05 to about 0.5 mg of monepantel/kg of body weight of the mammal. Alternatively, the effective amount of monepantel may be a daily or a substantially daily dose of from about 0.125 to about 0.5 mg/kg of body weight of the mammal. Alternatively, the effective amount of monepantel may be a daily or a substantially daily dose of from about 0.125 to about 0.25 mg of monepantel/kg of body weight of the mammal.

Animal feeds will typically contain from 0.00005 to about 0.0001 percent of monepantel (by weight) in the feed, preferably between about 0.0001 to about 0.005 percent of monepantel (by weight) in the feed, and most preferably between about 0.0005 to about 0.001 percent of monepantel component or components (by weight) in the feed.

In one aspect, this disclosure relates to a method of controlling a parasitic worm infection in a mammal by administering a systemically active oral composition including an anthelmintic, or a physiologically acceptable derivative or salt thereof, and animal feed at least once per week, more preferably three times per week, most preferably substantially daily.

In another aspect, this disclosure relates to a systemically active oral composition that includes an anthelmintic and animal feed.

This disclosure also relates to the use of an anthelmintic for the manufacture of an animal feed for controlling a parasitic worm infection in a mammal.

This disclosure also relates to a method of controlling a parasitic worm infection in a mammal for a prolonged time, comprising orally administering daily or substantially daily doses of an effective amount of an anthelmintic to the mammal in a daily feed. A daily feed is a feed that is intended to be administered daily, but which may be administered for effective times, as described herein. This method is especially useful for controlling parasitic worms in a mammal for a prolonged time comprising orally administering substantially daily doses of an effective amount of an anthelmintic to the mammal.

An aspect of this disclosure is the oral administration of an amount of an anthelmintic which is ineffective or sub-optimal for controlling parasitic worm infection in a mammal if administered at longer intervals, such as once per month. However, with repeated administrations at shorter intervals, e.g., substantially daily, as described herein, efficacious control of parasitic worm infections is achieved. Ineffective or sub-optimal means that a single dosing, as well as several dosings, results in less than a 50% reduction in the parasitic worm infestation, including no, or substantially no, reduction, as compared to no drug administration at all. This reflects the chronic, rather than acute, administration aspect disclosed herein.

In a first embodiment, a method is disclosed for controlling parasitic worms in a mammal by orally administering to the mammal a daily feed that includes an effective amount of an anthelmintic for an effective time.

In a second embodiment, an anthelmintic for use in controlling parasitic worms on a mammal in need thereof is disclosed. The anthelmintic may be present in a daily feed in an effective amount, said daily feed to be administered to said mammal for an effective time to control parasitic worms.

In a third embodiment, a daily feed for controlling parasitic worms in a mammal, comprising an effective amount of an anthelmintic when administered for an effective time is disclosed.

In an aspect of any of the embodiments, the mammal may be a canine. More particularly, the canine may be a wolf, coyote or a dog.

In an aspect of any of the embodiments, the mammal may be a feline. More particularly, the feline may be a domestic cat, bobcat, wildcat, ocelot, member of the genus lynx, Pallas's cat or cougar.

In an aspect of any of various embodiments, the anthelmintic may be fenbendazole and/or pyrantel pamoate and/or monepantel a salt thereof or mixture thereof. In some aspects other additional anthelmintics may be included in the inventive compositions and/or medicated feeds.

### EXAMPLES

The following non-limiting examples illustrate some of the methods of this disclosure:

### Example 1

Efficacy of pyrantel pamoate and fenbendazole Administered per os, i.e. by Mouth, to Dogs for the Treatment and Control of Ancylostoma caninum.

Methods: A pool of 22 dogs are to be preliminarily examined to ensure suitability for the example. Dogs must be within normal weight ranges for breed, in good health with no known history of serious disease and neither pregnant nor lactating. Between Day-7 and Day-3, a fecal examination may be performed to confirm the absence of pre-existing infection by any nematode species. Dogs exposed to an anthelmintic within 30 days prior to Day-7 are to be excluded.

Dogs are to be weighed on Day-2 and randomly assigned to 1 of 5 treatment groups by weight.

Dogs may be pair housed, if compatible, or individually housed until allocation to treatment groups on Day 0. Thereafter, the dogs are to be housed individually. Dogs are to be fed a standard commercially available dog food at the recommended rates and should be allowed access to potable water ad libitum.

Each dog in a treatment group (test groups 2-5) is to receive by mouth a liquid formulation of an anthelmintic. The dosage is to be administered to the dogs on each of days 0-22 according to test groups shown in TABLE 1.

TABLE 1

| | | | Anticipated | | |
|---|---|---|---|---|---|
| | | | Daily Dosage | | |
| | | | (mg active | | |
| | | | ingredient/kg | | |
| | # | Treatment | body wt of | | |
| Group | Dogs | Description | mammal)* | Formulation | Route |
| 1 | 4 | Non-treated, A. | 0 mg/kg | n/a | n/a |
| | | caninum | | | |
| | | infected control | | | |
| 2 | 4 | Pyrantel | 0.5 mg/kg daily | API in liquid | Oral |
| | | pamoate | for 23 | formulation | |
| | | | consecutive days | | |
| | | | (Days 0-22) | | |
| 3 | 4 | Pyrantel | 1.0 mg/kg daily | API in liquid | Oral |
| | | pamoate | for 23 | formulation | |
| | | | consecutive days | | |
| | | | (Days 0-22) | | |
| 4 | 4 | Fenbendazole | 5.0 mg/kg daily | API in liquid | Oral |
| | | | for 23 | formulation | |
| | | | consecutive days | | |
| | | | (Days 0-22) | | |
| 5 | 4 | Fenbendazole | 10.0 mg/kg daily | API in liquid | Oral |
| | | | for 23 | formulation | |
| | | | consecutive days | | |
| | | | (Days 0-22) | | |

*The selected dosages represent 10% and 20% of the therapeutic dosages for pyrantel pamoate (approx. 5 milligrams of the anthelmintic per kilogram of the mammal's body weight (mg/kg)) and fenbendazole ((approx. 50 milligrams of the anthelmintic per kilogram of the mammal's body weight (mg/kg))), respectively, to treat dogs for adult intestinal nematode infections as per label indications for existing commercial drug products. Current practice in the industry is to treat mammals with intestinal helminth with 3 separate 50 mg/kg dose of fenbendazole administered over a period of 3 days; accordingly under the current standard of care, the total effective monthly dose of fendbendzaole is about 150 mg/kg.

Dogs in the control group are not to receive any anthelmintics or any other worm control treatment. Dogs are to be fasted overnight. Each dog in treatment groups 2-5 is to be offered its daily morning ration (dry food) and the individual doses of liquid formulation are to be administered after the individual dog has eaten approximately 25% of its total morning ration. After receiving the dose of anthelmintic, the dogs are to be allowed to continue eating. This mimics incorporating the anthelmintic in feed.

Dogs are to be inoculated orally with approximately 300 infective, third-stage A. caninum larvae on Day 0, approximately 8 hours after the initial dose of anthelmintic. Fecal egg counts are to be performed on days 10, 13, 16, 19 and 22. There should also be a 7-day washout period from Day 23-Day 30. During the washout period, fecal egg counts should be performed on days 25, 28 and 30.

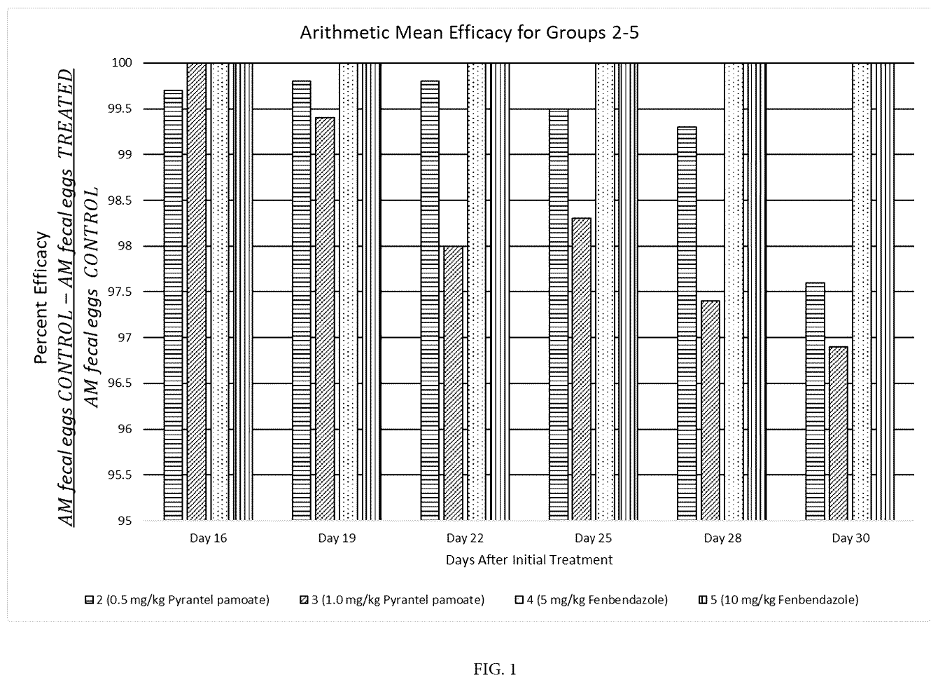

Percent reduction in fecal egg counts for the treatment groups are shown in the graph of FIG. 1. (Note that eggs were not recovered in any group for the fecal egg counts on days 10 and 13. Thus, these days are omitted from the chart in FIG. 1.)

### Example 2

Efficacy of pyrantel pamoate and fenbendazole Administered per os, i.e. by Mouth, to Dogs for the Treatment and Control of Toxocara canis.

Methods: A pool of 30 dogs are to be preliminarily examined to ensure suitability for the example. Dogs must be within normal weight ranges for breed, in good health with no known history of serious disease and neither pregnant nor lactating. On Day-6, a fecal examination may be performed to confirm the absence of pre-existing infection by any nematode species. Dogs exposed to an anthelmintic within 30 days prior to Day-7 are to be excluded.

28 eligible dogs are to be weighed on Day-1 and randomly assigned to 1 of 7 treatment groups by weight.

Dogs are to be housed indoors to help prevent naturally occurring parasitic worm infections. Dogs may be pair housed, if compatible, or individually housed until allocation to treatment groups on Day 0. Thereafter, the dogs are to be housed individually. Dogs are to be fed a standard commercially available dog food at the recommended rates and should be allowed access to potable water ad libitum.

Each dog in a treatment group (test groups 2-7) is to receive by mouth a liquid formulation of an anthelmintic. The dosage is to be administered to the dogs on each of days 0-52 according to test groups, see TABLE 2.

TABLE 2

| | | | Anticipated | | |
|---|---|---|---|---|---|
| | # | Treatment | Daily Dosage | | |
| Group | Dogs | Description | (mg/kg)* | Formulation | Route |
| 1 | 4 | Non-treated, T. | 0 mg/kg | n/a | n/a |
| | | canis | | | |
| | | infected | | | |
| | | control | | | |
| 2 | 4 | Pyrantel | 0.25 mg/kg daily | API in liquid | Oral |
| | | pamoate | for 53 | formulation | |
| | | | consecutive days | | |
| | | | (Days 0-52) | | |
| 3 | 4 | Pyrantel | 0.5 mg/kg daily | API in liquid | Oral |
| | | pamoate | for 53 | formulation | |
| | | | consecutive days | | |
| | | | (Days 0-52) | | |
| 4 | 4 | Pyrantel | 1.0 mg/kg daily | API in liquid | Oral |
| | | pamoate | for 53 | formulation | |
| | | | consecutive days | | |
| | | | (Days 0-52) | | |
| 5 | 4 | Fenbendazole | 2.5 mg/kg daily | API in liquid | Oral |
| | | | for 53 | formulation | |
| | | | consecutive days | | |
| | | | (Days 0-52) | | |
| 6 | 4 | Fenbendazole | 5.0 mg/kg daily | API in liquid | Oral |
| | | | for 53 | formulation | |
| | | | consecutive days | | |
| | | | (Days 0-52) | | |
| 7 | 4 | Fenbendazole | 10.0 mg/kg daily | API in liquid | Oral |
| | | | for 53 | formulation | |
| | | | consecutive days | | |
| | | | (Days 0-52) | | |

Dogs in the control group are not to receive any anthelmintics or any other worm control treatment. Dogs are to be fasted overnight. Each dog in treatment groups 2-7 is to be offered its daily morning ration (dry food) and the individual doses of liquid formulation are to be administered after the individual dog has eaten approximately 25% of its total morning ration. After receiving the dose of anthelmintic, the dogs are to be allowed to continue eating. This mimics incorporating the anthelmintic in feed.

Dogs are to be inoculated orally with approximately 250 infective, larvated eggs of T. canis on Day 0, approximately 8 hours after the initial dose of anthelmintic. Fecal egg counts are to be performed on days 30, 35, 40, 45, 50 and 52. There should also be a 7-day washout period from Day 53-Day 60. Following the washout period, fecal egg counts should be performed on day 60.

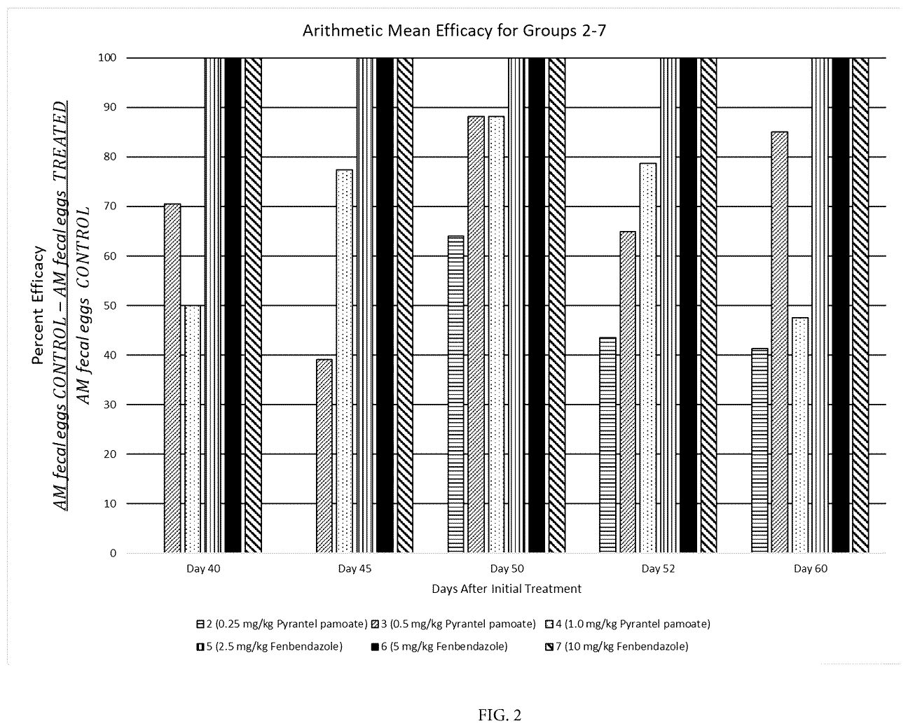

Percent reduction in fecal egg counts for the treatment groups are shown in the FIG. 2. (Note that no fecal eggs were recovered at the Day 30 and Day 35 counts. These days have been omitted from the graph of FIG. 2. Further, where more eggs were recovered in the fecal counts of the treatment group than in the fecal counts for the control group, no efficacy data is shown in FIG. 2 for that treatment group on that day.

### Example 3

Efficacy of Monepantel Administered per os, i.e. by Mouth, to Dogs for the Treatment and Control of Ancylostoma caninum.

Methods: A pool of 12 dogs are to be preliminarily examined to ensure suitability for the example. Dogs must be within normal weight ranges for breed, in good health with no known history of serious disease and neither pregnant nor lactating. Between Day-7 and Day-3, a fecal examination may be performed to confirm the absence of pre-existing infection by any nematode species. Dogs exposed to an anthelmintic within 30 days prior to Day-7 are to be excluded.

Dogs are to be weighed on Day-2 and randomly assigned to 1 of 3 groups by weight (1 control group and 2 treatment groups).

Dogs may be pair housed, if compatible, or individually housed until allocation to treatment groups on Day 0. Thereafter, the dogs are to be housed individually. Dogs are to be fed a standard commercially available dog food at the recommended rates and should be allowed access to potable water ad libitum.

Each dog in a treatment group (test groups 2-3) is to receive by mouth a liquid formulation of monepantel. The dosage is to be administered to the dogs on each of days 0-22 according to test groups, see TABLE 3.

TABLE 3

| | | | Anticipated | | |
|---|---|---|---|---|---|
| | # | Treatment | Daily Dosage | | |
| Group | Dogs | Description | (mg/kg) | Formulation | Route |
| 1 | 4 | Non-treated, A. | 0 mg/kg | n/a | n/a |
| | | caninum | | | |
| | | infected | | | |
| | | control | | | |
| 2 | 4 | Monepantel | 0.25 mg/kg daily | API in liquid | Oral |
| | | | for 23 | formulation | |
| | | | consecutive days | | |
| | | | (Days 0-22) | | |
| 3 | 4 | Monepantel | 0.5 mg/kg daily | API in liquid | Oral |
| | | | for 23 | formulation | |
| | | | consecutive days | | |
| | | | (Days 0-22) | | |

Dogs in the control group are not to receive any anthelmintics or any other worm control treatment. Dogs are to be fasted overnight. Each dog in treatment groups 2-3 is to be offered its daily morning ration (dry food) and the individual doses of liquid formulation are to be administered after the individual dog has eaten approximately 25% of its total morning ration. After receiving the dose of anthelmintic, the dogs are to be allowed to continue eating. This mimics incorporating the anthelmintic in feed.

Dogs are to be inoculated orally with approximately 300 infective, third-stage A. caninum larvae on Day 0, approximately 8 hours after the initial dose of anthelmintic. Fecal egg counts are to be performed on days 10, 13, 16, 19 and 22. There should also be a 7-day washout period from Day 23-Day 30. During the washout period, fecal egg counts should be performed on days 25, 28 and 30.

Percent reduction in fecal egg counts for the treatment groups are expected to achieve 100% within approximately 2 weeks.

### Example 4

Efficacy of Monepantel Administered per os, i.e. by Mouth, to Dogs for the Treatment and Control of Toxocara canis.

Methods: A pool of dogs are to be preliminarily examined to ensure suitability for the example. Dogs must be within normal weight ranges for breed, in good health with no known history of serious disease and neither pregnant nor lactating. On Day-6, a fecal examination may be performed to confirm the absence of pre-existing infection by any nematode species. Dogs exposed to an anthelmintic within 30 days prior to Day-7 are to be excluded.

16 eligible dogs are to be weighed on Day-1 and randomly assigned to 1 of 4 groups by weight (1 control group and 3 treatment groups).

Dogs are to be housed indoors to help prevent naturally occurring parasitic worm infections. Dogs may be pair housed, if compatible, or individually housed until allocation to treatment groups on Day 0. Thereafter, the dogs are to be housed individually. Dogs are to be fed a standard commercially available dog food at the recommended rates and should be allowed access to potable water ad libitum.

Each dog in a treatment group (test groups 2-4) is to receive by mouth a liquid formulation of monepantel. The dosage is to be administered to the dogs on each of days 0-52 according to test groups, see TABLE 4.

TABLE 4

| | | | Anticipated | | |
|---|---|---|---|---|---|
| | # | Treatment | Daily Dosage | | |
| Group | Dogs | Description | (mg/kg)* | Formulation | Route |
| 1 | 4 | Non-treated, T. | 0 mg/kg | n/a | n/a |
| | | canis infected | | | |
| | | control | | | |
| 2 | 4 | Monepantel | 0.125 mg/kg daily | API in liquid | Oral |
| | | | for 53 | formulation | |
| | | | consecutive days | | |
| | | | (Days 0-52) | | |
| 3 | 4 | Monepantel | 0.25 mg/kg daily | API in liquid | Oral |
| | | | for 53 | formulation | |
| | | | consecutive days | | |
| | | | (Days 0-52) | | |
| 4 | 4 | Monepantel | 0.5 mg/kg daily | API in liquid | Oral |
| | | | for 53 | formulation | |
| | | | consecutive days | | |
| | | | (Days 0-52) | | |

Dogs in the control group are not to receive any anthelmintics or any other worm control treatment. Dogs are to be fasted overnight. Each dog in treatment groups 2-4 is to be offered its daily morning ration (dry food) and the individual doses of liquid formulation are to be administered after the individual dog has eaten approximately 25% of its total morning ration. After receiving the dose of anthelmintic, the dogs are to be allowed to continue eating. This mimics incorporating the anthelmintic in feed.

Dogs are to be inoculated orally with approximately 250 infective, larvated eggs of T. canis on Day 0, approximately 8 hours after the initial dose of anthelmintic. Fecal egg counts are to be performed on days 30, 35, 40, 45, 50 and 52. There should also be a 7-day washout period from Day 53-Day 60. Following the washout period, fecal egg counts should be performed on day 60.

100% efficacy is expected for all treatment groups by the end of the study period.

### Example 5

Efficacy of Fenbendazole Administered per os, i.e. by Mouth, to Dogs for the Treatment and Control of Toxocara canis Infections.

Methods: A pool of dogs are to be preliminarily examined to ensure suitability for the example. Dogs must be within normal weight ranges for breed, in good health with no known history of serious disease and neither pregnant nor lactating. Between Day-6 and Day-3, a fecal examination may be performed to confirm the absence of pre-existing infection by any nematode species. Dogs exposed to an anthelmintic within 30 days prior to Day-7 are to be excluded.

20 eligible dogs are to be weighed on Day-2 or Day-1 and randomly assigned to 1 of 5 groups by weight (1 control group and 4 treatment groups).

Dogs are to be housed indoors to help prevent naturally occurring parasitic worm infections. Dogs may be pair housed, if compatible, or individually housed until allocation to treatment groups on Day 0. Thereafter, the dogs are to be housed individually. Dogs are to be fed a standard commercially available dog food at the recommended rates and should be allowed access to potable water ad libitum.

Each dog in a treatment group (test groups 2-5) is to receive by mouth a liquid formulation of fenbendazole. The dosage is to be administered to the dogs on each of days 0-52 according to test groups, see TABLE 5.

TABLE 5

| | | | Anticipated | | |
|---|---|---|---|---|---|
| | # | Treatment | Daily Dosage | | |
| Group | Dogs | Description | (mg/kg)* | Formulation | Route |
| 1 | 4 | Non-treated, | 0 mg/kg | n/a | n/a |
| | | T. canis | | | |
| | | infected | | | |
| | | control | | | |
| 2 | 4 | Fenbendazole | 0.5 mg/kg daily | FBZ in | Oral |
| | | | for 53 | liquid | |
| | | | consecutive days | formulation | |
| | | | (Days 0-52) | | |
| 3 | 4 | Fenbendazole | 1.0 mg/kg daily | FBZ in | Oral |
| | | | for 53 | liquid | |
| | | | consecutive days | formulation | |
| | | | (Days 0-52) | | |
| 4 | 4 | Fenbendazole | 1.5 mg/kg daily | FBZ in | Oral |
| | | | for 53 | liquid | |
| | | | consecutive days | formulation | |
| | | | (Days 0-52) | | |
| 5 | 5 | Fenbendazole | 0.5 mg/kg daily | FBZ in | Oral |
| | | | for 53 | experimental | |
| | | | consecutive days | liquid | |
| | | | (Days 0-52) | formulation | |

Dogs in the control group are not to receive any anthelmintics or any other worm control treatment. Dogs are to be fasted overnight. Each dog in treatment groups 2-5 is to be offered its daily morning ration (dry food) and the individual doses of liquid formulation are to be administered after the individual dog has eaten approximately 25% of its total morning ration. After receiving the dose of anthelmintic, the dogs are to be allowed to continue eating. This mimics incorporating the anthelmintic in feed.

Dogs are to be inoculated orally with approximately 250 infective eggs of T. canis on Day 0, approximately 8 hours after the initial dose of anthelmintic. Fecal egg counts are to be performed on days 30, 35, 40, 45, 50 and 53.

100% efficacy is expected for at least treatment groups 2-4 by the end of the study period.

While this invention has been described as having an exemplary design, the present invention may be further modified within the spirit and scope of this disclosure. This application is therefore intended to cover any variations, uses, or adaptations of the invention using its general principles.

What is claimed is:

1. A medicated animal feed for controlling intestinal helminths in a mammal, comprising:
a feedstuff; and
a dose of at least one anthelmintic that:
(i) disrupts cellular integrity in intestinal helminths and is selected from the group consisting of benzimidazoles, salicylanilides, and substituted phenyls, and/or
(ii) causes paralysis in intestinal helminths and is selected from the group consisting of tetrahydropyrimidines, emodepside, and monepantel;
wherein the at least one anthelmintic is an integral part of the medicated animal feed and the effective time to achieve a therapeutically effective level of the anthelmintic in the mammal's digestive tract is selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

2. The medicated animal feed according to claim 1, wherein the mammal is selected from a group consisting of: canines and felines.

3. The medicated animal feed according to claim 1, wherein the intestinal helminth is at least one intestinal helminth selected from the group consisting of: Toxocara canis, Toxascaris leonina, Ancylostoma caninum, Ancylostoma braziliensis, Uncinaria stenocephala, Trichuris vulpis, Toxocara cati, Ancylostoma tubaeforme, Taenia spp., Dipylidium spp. and Echinococcus spp.

4. The medicated animal feed according to claim 1, wherein the anthelmintic is pyrantel or a therapeutically acceptable derivative, salt or metabolite thereof, and wherein the amount of pyrantel in the medicated feed is such that the total therapeutic dose of pyrantel does not exceed about 50.0 mg of pyrantel per kg of the mammal's body mass (mg/kg) when administered to the mammal in a plurality of doses within a period of time of about 30 days.

5. The medicated animal feed according to claim 4, wherein the anthelmintic is pyrantel pamoate.

6. The medicated animal feed according to claim 4, wherein the daily dose of pyrantel in the medicated feed is selected from the group consisting of: about 0.1 mg/kg, about 0.25 mg/kg, about 0.5 mg/kg, and about 1.0 mg/kg.

7. The medicated animal feed according to claim 1 wherein the anthelmintic is monepantel or a therapeutically acceptable derivative, salt or metabolite thereof, and wherein the amount of monepantel in the medicated feed is such that the total therapeutic dose of monepantel does not exceed about 25.0 mg of monepantel per kg of the mammal's body mass (mg/kg) when administered to the mammal in a plurality of doses within a period of time of about 30 days.

8. The medicated animal feed according to claim 7, wherein the daily dose of monepantel in the medicated feed is selected from the group consisting of: about 0.05 mg/kg, about 0.125 mg/kg, about 0.25 mg/kg, about 0.5 mg/kg, about 0.83 mg/kg.

9. The medicated animal feed according to claim 1, wherein the anthelmintic in the medicated feed that disrupts cellular integrity in the intestinal helminths is a benzimidazole.

10. The medicated animal feed according to claim 1, wherein the anthelmintic is fenbendazole or a therapeutically acceptable derivative, salt or metabolite thereof, and wherein the amount of fenbendazole in the medicated feed is such that the total therapeutic dose of fenbendazole does not exceed about 300 mg of fenbendazole per kg of the mammal's body mass (mg/kg) when administered to the mammal in a plurality of doses within a period of time of about 30 days.

11. The medicated animal feed according to claim 10, wherein the daily dose of fenbendazole in the medicated feed is selected from the group consisting of: about 0.1 mg/kg, about 0.25 mg/kg, about 0.5 mg/kg, about 1.0 mg/kg, about 2.5 mg/kg, about 5.0 mg/kg, and about 10 mg/kg.

12. The medicated animal feed according to claim 1, wherein the medicated animal feed further comprises a therapeutically effective amount of praziquantel or a therapeutically acceptable derivative, salt or metabolite thereof.

13. A method for controlling intestinal helminths in a mammal, comprising the steps of:
providing the medicated feed according to claim 1; and
administering said feed to a mammal on a daily or a substantially daily basis.

14. The method according to claim 13, wherein the medicated feed is administered over a period selected from the group consisting of: more than 1 day but less than 20 days, more than 20 days but less than 30 days, more than 30 days less than 40 days, more than 40 days but less than 50 days, more than 50 days but less than 60 days, and more than 60 days, when the medicated animal feed is administered to the mammal daily or substantially daily.

15. A composition for controlling intestinal helminths in a mammal, comprising:
at least one anthelmintic that:
(i) disrupts cellular integrity in intestinal helminths and is selected from the group consisting of benzimidazoles, salicylanilides, and substituted phenyls, and/or
(ii) causes paralysis in intestinal helminths and is selected from
the group consisting of tetrahydropyrimidines, emodepside, and monepantel;
wherein the effective time to achieve a therapeutically effective level of the anthelmintic in the mammal's digestive tract is selected from the group consisting of: more than 1 day but less than 20 days, more than 20 days but less than 30 days, more than 30 days less than 40 days, more than 40 days but less than 50 days, more than 50 days but less than 60 days, and more than 60 days, when the composition is administered to the mammal daily or substantially daily.

16. The composition according to claim 15, wherein the composition is selected from the group consisting of: a kibble, a wet feed, a tablet, a capsule, a suspension, a solution, a salve, a paste, a treat, and a snack.

17. The composition according to claim 15, wherein the composition is suitable to administer to the mammal on a daily or substantially daily basis over the period of time.

18. The composition according to claim 15, wherein the anthelmintic is pyrantel or a therapeutically acceptable derivative, salt or metabolite thereof, and wherein the amount of pyrantel in the composition is such that the total therapeutic dose of pyrantel does not exceed about 50.0 mg of pyrantel per kg of the mammal's body mass (mg/kg) when administered to the mammal in a plurality of doses within a period of time of about 30 days.

19. The composition according to claim 15 wherein the anthelmintic is monepantel or a therapeutically acceptable derivative, salt or metabolite thereof, and wherein the amount of monepantel in the composition is such that the total therapeutic dose of monepantel does not exceed about 25.0 mg of monepantel per kg of the mammal's body mass (mg/kg) when administered to the mammal in a plurality of doses within a period of time of about 30 days.

20. The composition according to claim 15, wherein the anthelmintic in the composition that disrupts cellular integrity in the intestinal helminths is a benzimidazole.
